(19) United States
(12) Patent Application Publication
(10) Pub. No.: US 2019/0156948 A1
(43) Pub. Date: May 23, 2019

(54) MANAGEMENT APPARATUS AND SYSTEM

(71) Applicant: Paramount Bed Co., Ltd., Tokyo (JP)

(72) Inventor: Takuro HATSUKARI, Tokyo (JP)

(73) Assignee: Paramount Bed Co., Ltd., Tokyo (JP)

(21) Appl. No.: 16/193,078

(22) Filed: Nov. 16, 2018

(30) Foreign Application Priority Data
Nov. 20, 2017 (JP) 2017-222969

(51) Int. Cl. G16H 50/20; G16H 20/10

(52) U.S. Cl. CPC G16H 50/20; G16H 20/10

(57) ABSTRACT

A management apparatus includes, a memory configured to store treatment information on a patient, an interface unit configured to communicate with an external device, and a controller configured to obtain the treatment information from the memory, determine a proposal for the patient, based on the treatment information, and order equipment selected based on the proposal to the external device.

### CROSS-REFERENCE TO RELATED APPLICATIONS

This nonprovisional application claims priority under 35 U.S.C. § 119 (a) on Patent Application No. 2017-222969 filed in Japan on 20 Nov. 2017, the entire contents of which are hereby incorporated by reference.

### FIELD

The present embodiment relates to a management apparatus and a system.

### BACKGROUND

Various inventions have hitherto been devised to prevent users of beds such as patients from falling down to the floor from the beds.

### BRIEF DESCRIPTION OF THE DRAWINGS

FIG. 1 is a diagram for explaining the entire system and functional components in a first embodiment;

FIG. 2 is a diagram for explaining an example of a data table of equipment management information in the first embodiment;

FIG. 3 is a diagram for explaining components of a management apparatus according to the first embodiment;

FIG. 4 is a flowchart for illustrating a first process of the management apparatus according to the first embodiment;

FIG. 5 is a flowchart for illustrating a second process in the first embodiment;

FIG. 6 is a flowchart for illustrating a third process in the first embodiment;

FIG. 7 is a sequence diagram for explaining flows between a management apparatus and servers in the first embodiment;

FIG. 8 is a flowchart for illustrating a first process of a management apparatus according to a second embodiment; and

FIG. 9 is a flowchart for illustrating a first process of a management apparatus in the third embodiment.

### DETAIL DESCRIPTION

One or more embodiments are now described with reference to the drawings, wherein like reference numerals are used to refer to like elements throughout. In the following description, for purposes of explanation, numerous specific details are set forth in order to provide a thorough understanding of the various embodiments. It is evident, however, that the various embodiments can be practiced without these specific details (and without applying to any particular networked environment or standard).

As used in this disclosure, in some embodiments, the terms “component,” “system” and the like are intended to refer to, or comprise, a computer-related entity or an entity related to an operational apparatus with one or more specific functionalities, wherein the entity can be either hardware, or a combination of hardware and software in execution.

One or more components may reside within a process and/or thread of execution and a component maybe localized on one computer and/or distributed between two or more computers. In addition, these components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software application or firmware application executed by a processor, wherein the processor can be internal or external to the apparatus and executes at least a part of the software or firmware application. As yet another example, a component can be an apparatus that provides specific functionality through electronic components without mechanical parts, the electronic components can comprise a processor therein to execute software stored on a non-transitory electronic memory or firmware that confers at least in part the functionality of the electronic components. While various components have been illustrated as separate components, it will be appreciated that multiple components can be implemented as a single component, or a single component can be implemented as multiple components, without departing from example embodiments. Further, the various embodiments can be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer-readable (or machine-readable) device or computer-readable (or machine-readable) storage/communications media having a computer program stored thereon. For example, computer readable storage media can comprise, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

In addition, the words “example” and “exemplary” are used herein to mean serving as an instance or illustration. Any embodiment or design described herein as “example” or “exemplary” is not necessarily to be construed as preferred or advantageous over other embodiments or designs. Rather, use of the word example or exemplary is intended to present concepts in a concrete fashion. As used in this application, the term “or” is intended to mean an inclusive “or” rather than an exclusive “or”. That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Embodiments described herein can be exploited in substantially any wireless communication technology, comprising, but not limited to, wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Z-Wave, Zigbee and other 802.XX wireless technologies and/or legacy telecommunication technologies.

In general, one aspect of the present application is A management apparatus includes, a memory configured to store treatment information on a patient, an interface unit configured to communicate with an external device, and a controller configured to obtain the treatment information from the memory, determine a proposal for the patient, based on the treatment information, and order equipment selected based on the proposal to the external device.

Hereinafter, the present embodiment will be described with reference to the drawings. In the preceding examples, although various ways against falling down from the beds have been considered, these ways have not been systematically applied. It has not been known what ways are effective against accidentally falling down from the beds.

A possibility that the patient tumbles and falls from a bed varies depending on whether the patient have taken any surgery, medication, and the like. But it is difficult to make an appropriate proposal in accordance with the possibility and it is necessary for caregivers such as medical staffs to make any proposals to reduce the risk of falling down from the bed every time, which causes a big burden to the caregivers.

The present embodiment discloses a system that can propose an appropriate apparatus and measures for the user of the bed, but is not limited to the system. The system of the embodiment is usually used in the hospital for example. However, when the system is used in a regional comprehensive care system, it is possible to propose an appropriate apparatus and measures for the user, with communication between the server in the hospital and the server of a clinic which is different location from the hospital. Further, the system can also be applied to systems in which a plurality of facilities and places cooperate, such as a collaboration system between a plurality of facilities for the elderly, and a collaboration system between a system in a hospital and a system in a dispensing pharmacy.

In addition, in the description, the term “patient” refers to a person using a bed (or a mattress), and is not limited to a person who receives medical treatment for illness, but also a person who receives nursing care at a facility or a person who goes to a bed can be regarded as the patient.

### 1. First Embodiment

### [1.1 System Configuration]

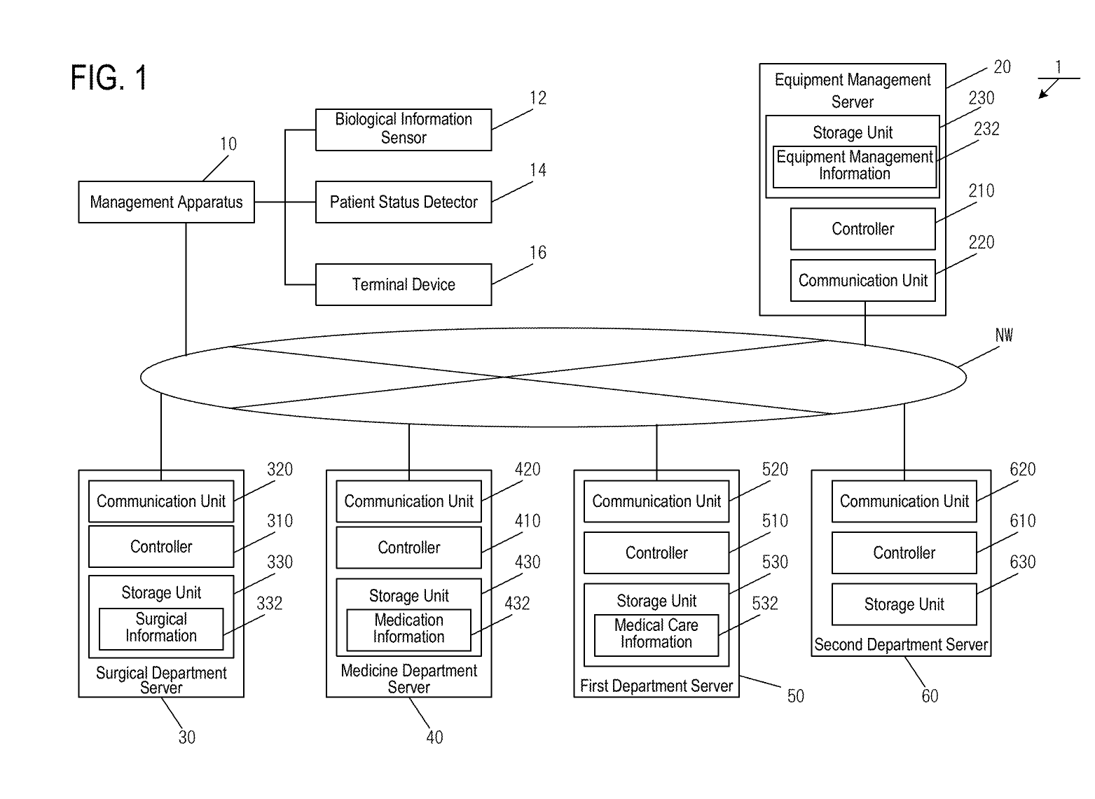

Referring first to FIG. 1, the entire system and the components of the present system will be described. FIG. 1 is a diagram for explaining the entire system 1 and the configuration of each component. The system 1 has a management apparatus 10; an equipment management server 20; a surgical department server 30; a medicine department server 40; and other department servers including a first department server 50 and a second department server 60. These apparatus and servers can communicate with a network NW. Here, for example, other additional servers such as accounting server and examination server may communicate with the network NW.

The management apparatus 10 manages information on patients or facilities in this embodiment. Information on patients includes basic information on patients such as name, gender and age; medical information such as disease name and biological data; and information on treatment and medicine. The management apparatus 10 is configured to allow a user to input the information on the patients and to view the information on the patients.

In addition, based on the information on the patients, the management apparatus 10 can propose the ways against accidentally tumbling and falling down from the beds in accordance with the standardized flow in the hospital, facility or the like. The ways involve some proposals to provide at least one of the sensors, welfare equipment, and the like. Based on some proposal, professionals such as doctors and caregivers can easily make final decisions whether the proposed way would be applied (for example, professionals approve use of proposed sensors, welfare equipment, etc.), whereby the management apparatus 10 can reduce the risk of falling down from the bed for the patient in real time.

The management apparatus 10 can communicate with the servers 30 to 60 via the network NW. Further, as shown in FIG. 1, the management apparatus 10 can communicate, for example, with a biological information sensor 12, a patient status detector 14 and a terminal device 16.

The biological information sensor 12 obtains biological information on a patient. The biological information sensor 12 obtains biological information on a patient through, for example, a sensor attached on the patient body or a sensor (device) disposed between sections of the bed and the mattress.

Examples of the sensors attached on the patient body include sensors directly attached on arms or chest by the caregivers or patients. The sensors may include, for example, infrared sensors, sensors that obtain weak electricity, sensors that detect vibration and the like. By using these sensors, the biological information sensor 12 can collect various kinds of information such as heartbeat (heartbeat waveform and pulse rate), respiration (respiration waveform and respiratory rate), body motion, etc. of the patient.

Further, the sensors provided between the sections of the bed and the mattress may include pressure detection sensors, load sensors, sound detection sensors and the like. By using these sensors, the biological information sensor 12 can collect heartbeat; respiration; body motion; whether the patient is sleeping or not; a depth of the sleeping of the patient; a posture or a position of the patient in the bed when the patient is sleeping and the like of the patient.

The biological information sensor 12 may collect biological information from measuring instruments for measuring the conditions and states of the patient every time the patient or the doctor measures the biological information. For example, the biological information sensor 12 may obtain the body temperature measured by a thermometer; the blood pressure measured by a sphygmomanometer; the blood glucose level measured by a glucometer; and others.

In addition, the biological information sensor 12 can collect biological information mainly in real time, but may periodically collect the biological information at some intervals. Further, although in the above description, the biological information sensor 12 is assumed to use sensors that directly or indirectly touch the patient, non-contact devices such as infrared sensors, cameras and the like installed at the side of the bed may be used to collect biological information.

The patient status detector 14 may be provided in the mattress, the bed or their surroundings to detect whether the patient is in the bed or not. The patient status detector 14 may be provided between the sections of the bed and the mattress. In addition, the patient status detector 14 can detect the posture and position of the patient when the patient is in bed. The patient status detector 14 can detect the state of the patient, e.g., getting out of bed, staying in bed, position, posture and others by using pressure sensors placed under the bed, infrared sensors disposed in or around the bed and/or load sensors provided in the bed.

Regarding to the sensors for detecting that whether the patient is in the bed or not, whether the patient is staying in bed, position of the patient, posture of the patient, and others, a state detecting method is described in Japanese Patent Application Laid-Open No. 2008-206869 (the title of the invention: abed, filing date: Feb. 27, 2007) and a user position detecting method is described in Japanese Patent Application Laid-Open No. 2009-118980 (the title of the invention: a system of detecting the state of a user in bed, filing date: Nov. 13, 2007). The entire contents of these patent applications are incorporated by reference.

It is also possible to detect whether the patient is getting out of the bed, position of the patient when the patient is staying in the bed and posture of the patient when the patient is staying in the bed in accordance with a pressure detected from sensors placed between the sections of the bed and the mattress. A method for detecting whether the patient is in the bed or not is described in Japanese Patent Application Laid-Open No. 2002-327624 (the title of the invention: a device of detecting in/out of bed, filing date: Nov. 11, 2002), a detecting method is described in Japanese Patent Application No. 2002-327632 (the title of the invention: a device for detecting a positional shift on the bed, filing date: Nov. 11, 2002), and a detecting method is described in Japanese Patent Application No. 2002-327633 (the title of the invention: a device for positional detection on the bed, filing date: Nov. 11, 2002). The entire contents of these patent applications are incorporated by reference.

The patient status detector 14 may be integrated with the above-described biological information sensor 12. For example, the sensors placed between the sections of the bed and the mattress may obtain biological information. Likewise, the patient status detector 14 can detect the conditions and status of the patient by using the sensors.

The terminal device 16 displays various kinds of information for a patient and caregivers. For example, the terminal device 16 is placed at the side of the bed, and displays information on the patient and displays biological information on the patient. Further, in the present embodiment, the terminal device 16 and the management apparatus 10 are described as separate components, but may be integrated.

The equipment management server 20 manages various kinds of equipment. The term “equipment” used herein refers to general equipment available to patients, such as measuring instruments and therapeutic equipment. The equipment may include a monitor which can detect the biological information; devices directly used by the patient (e.g., respirator); sensors (e.g., bed-exit/staying detecting sensor); welfare equipment (e.g., wheelchair or walker); assisting devices which are mountable or attachable to the bed, and the like.

The network NW can communicate with the equipment management server 20, the surgical department server 30, the medicine department server 40, the first department server 50 and the second department server 60 through respective communication units.

The communication unit (communication units 220, 320, 420, 520, 620) in each server 20, 30, 40, 50, 60 communicates with other servers and devices through the network NW. The communication unit may communicate with other servers and devices through the network NW via a wired LAN, a wireless LAN, or a public line used for a LTE.

The controller (controllers 210, 310, 410, 510, and 610) in each server 20, 30, 40, 50, 60 controls the server as a whole. The controller controls the server by reading out and executing various programs stored in the storage unit (storage units 230, 330, 430, 530, 630) provided in each server 20, 30, 40, 50, 60. The controller is constituted by a CPU (Central Processing Unit), for example.

The storage unit (storage units 230, 330, 430, 530, 630) in each server 20, 30, 40, 50, 60 stores various programs and various data necessary for the operation of the server. The storage unit is composed of, for example, an SSD (Solid State Drive) which is a semiconductor memory, an HDD (Hard Disk Drive) which is a magnetic disk, and the like.

Here, the equipment management information 232 for managing equipment is stored in the equipment management server 20.

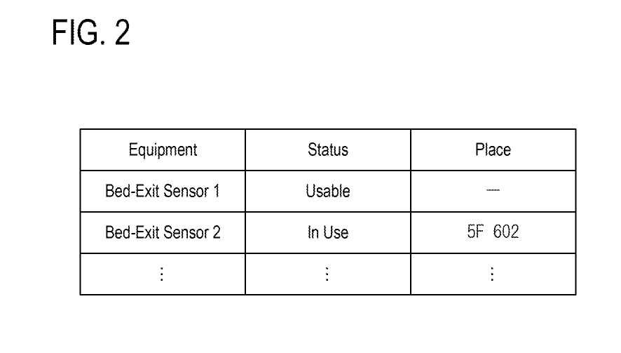

The storage unit 230 stores information regarding the equipment being managed by the equipment management server 20. Here, FIG. 2 shows an example of a data table of the equipment management information 232.

As shown in FIG. 2, in the equipment management information 232, the equipment name (e.g., name of sensor which can detect whether the patient is in the bed, the sensor is called as “bed-exit sensor 2”) is stored in association with the status of the equipment (e.g., whether the equipment is used, if patient use the bed-exit sensor 2, we define the status of the equipment as “in use”) and the place of the equipment (e.g., “5F 602” which indicates the place where the bed-exit sensor 2 is used).

The sensors managed by the equipment management information 232 may include detector which evaluates whether the patient is sleeping, detector which can detect whether the patient gets out of the bed, mat sensors, string sensors, infrared sensors and the like. Also, the welfare equipment managed by the equipment management information 232 may include bed whose sections can be relatively low height, electrical bed, assisting devices, handrails which can attach with the bed, cushioning mat which can attach with the bed and the like.

Here, the status stored in the equipment management information 232 may indicate whether the equipment is currently “in use”, “usable” or “unusable”. Also, the place stored in the equipment management information 232 may indicate where the equipment is currently used. In FIG. 2, the place stored in the equipment management information 232 indicates the location of the patient's room, but it may be any information as long as the location of the equipment can be specified, such as the patient's identification (ID) information.

The servers 30 to 60 manage information on patients in respective departments in the hospital. The servers in this embodiment in FIG. 1 include the surgical department server 30 that manages (stores) surgical information 332 as information on surgery; the medicine department server 40 that manages (stores) medicine information 432 as information on medicine to patients; and the first and second department servers 50 and 60 as other department servers.

The first department server 50 and the second department server 60 are used in departments other than the surgical department and the medicine department. For example, the first department server 50 may be an electronic medical record server in which electronic medical records on patient are stored. This server is used for a clinical department where patients are currently receiving medical treatment, and the server stores medical care information 532 which is information on medical care/treatment for patients. The second department server 60 in this case is used for the department other than the clinical department.

The clinical department in which a patient receives medical care is specified by the patient information stored in the electronic medical record, or by the patient ID stored in the first department server 50. Further, the patient ID can be specified, for example, by a patient registration card or an RFID built in an identification tag the patient has.

Further, the first department server 50 may store various kinds of information such as information on falling assessment on the patient and medical treatment environment assessment. The information indicates whether the possibility the patient accidentally falls down from the bed is relatively high, normal, or low.

The surgical information 332 is stored in the surgical department server 30 for each patient. The medicine information 432 is stored in the medicine department server 40 for each patient. The medical care information 532 is stored in the first department server 50 for each patient. That is, the surgical information 332 stores surgical details for each patient ID for identifying a patient. The medicine information 432 stores types, name, doses and the like of the prescribed medicine for each patient ID. Further, the medical care information 532 stores the status and others of each patient for each patient ID.

The surgical information 332 stored in the surgical department server 30 stores information on surgery in association with the patient ID. Information relating to surgical operations includes, for example, surgery schedule (date and time, place etc.), a doctor who is in charge (surgeon), an anesthesia record, a medical record before and after operation, nursing record and the like for each patient. The surgical information 332 may also include information on hospital such as an anesthesia ledger, surgery invoices and slips and the like. Referring to the surgical information 332, the surgical department server 30 can comprehensively manage preoperative, intraoperative, postoperative information on each patient, and can cooperate and communicate with other systems.

The medicine information 432 stored in the medicine department server 40 have information on medicine in association with each patient ID. The information on medicine includes, for example, information for the patient such as prescription, injection, history of medicine the patient brought, medicine guidance record, and the like. The medication information 432 may store also information on prescribed medicines such as data on the medicine (e.g. components of the medicine) and document of the medicine. Referring to the medicine information 432, the medicine department server 40 can comprehensively manage patient information including prescription, injection and diagnostic medicine, and the medicine department server 40 can cooperate and communicate with other systems.

The first department server 50 manages the medical treatment the patient is currently receiving. The first department server is, for example, an electronic medical record server that stores electronic medical records. The first department server 50 may be assigned to each medical department. In the case where a department server is assigned to each medical department, if the patient is hospitalized in the department of internal medicine, the server to be used in the department of internal medicine functions as the first department server 50. The medical care information 532 of this first department server 50 stores information on electronic medical records, patient information such as various examination results, and nursing information such as falling assessment and medical treatment environment assessment on each patient.

The first and second department servers 50 and 60 may be discriminated in other ways. For example, a department server may be allocated to each ward, each room or each medical department group. Further, though the present embodiment includes two department servers, i.e., the first department server 50 and the second department server 60, three or more department servers may be provided.

Alternatively, depending on the scale of a system, the system may have only one server which functions as a plurality of department servers.

Note that the system 1 described in FIG. 1 is an example of the description in this embodiment, and other configurations of the system 1 may be adopted. For example, although it has been described that a plurality of devices are collectively managed by the equipment management server 20, the plurality of devices may be separately managed by other servers.

Also, in a medical system, the system configuration usually may be further sub-divided. In the present embodiment, however, the necessary configuration is simplified for explanation. As one example, a system can be considered in which individual systems (servers) can communicate with an order system including an electronic medical record system. The systems which can communicate with the order system may be systems (servers) such as a pharmacy system, a medicine management guidance recording system, an ICU management system, an ME clinical system, a nursing support system, an equipment management system, a medical accounting system, a surgical management system and others.

The above-described various kinds of information are stored and managed as appropriate in each system as necessary. For example, when the nursing support system is in the system, nursing information such as each assessment is stored and managed in the nursing support system.

### [1.2 Configuration of Management Apparatus]

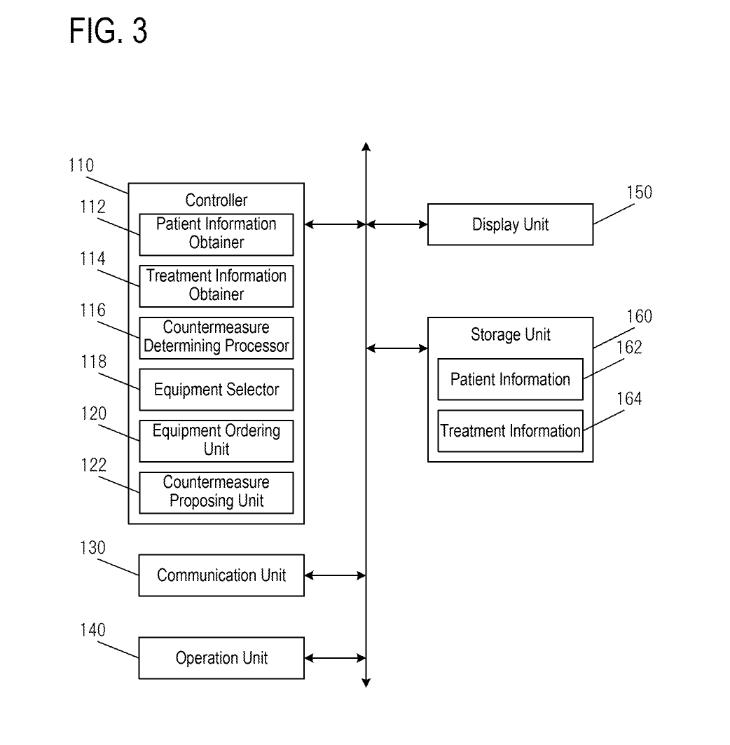

The configuration of the management apparatus 10 will be described with reference to FIG. 3. As shown in FIG. 3, the management apparatus 10 includes a controller 110, a communication unit 130, an operation unit (a user interface unit) 140, a display unit 150 and a storage unit 160.

The controller 110 controls the management apparatus 10 as a whole. The controller 110 controls the management apparatus 10 by reading out and executing various programs stored in the storage unit 160, and is configured of, for example, a CPU (Central Processing Unit).

The controller 110 reads out and executes the programs stored in the storage unit 160, to thereby perform functions of a patient information obtainer 112, a treatment information obtainer 114, a determining processor 116, an equipment selector 118, an equipment ordering unit 120 and a proposing unit 122.

The patient information obtainer 112 obtains the biological information obtained by the biological information sensor 12 and the status of the patient detected by the patient status detector 14 as information on the patient. That is, the patient information obtainer 112 can obtain biological information such as a heart rate of a patient and respiration of a patient, the state of patient (including whether the patient is sleeping or not, that is, a sleeping state, an awaken state), and information whether the patient is staying in bed or not, the patient's posture when the patient is in the bed and the patient's position when the patient is in the bed.

The patient information collected by the patient information obtainer 112 is stored in the storage unit 160 as patient information 162. Here, the patient information 162 typically stored can be listed as follows for example:
(1) Biological information such as heartbeat, respiration, etc. of the patient;
(2) Biological information such as heart rate and respiratory rate based on heartbeat, rate of breathing etc. of the patient;
(3) Measurement information obtained from measuring devices such as patient's body temperature, blood pressure, blood glucose level, etc.;
(4) information including whether the patient is sleeping or awaken, depth of sleeping, patient's posture and patient's position when the patient is staying in the bed;
(5) Sleeping time or period calculated from the information described in the item (4), time or period the patient got out of the bed and the number of times the patient got out of the bed; and
(6) Other information obtained from the biological information obtainer 12 and the patient status detector 14 and values calculated based on the obtained information.
   The patient information stored in the patient information 162 may be updated in real time, or may be stored accumulatively. Also, the patient information may be periodically collected and stored at some intervals (for example, every minute, every 30 minutes, every hour, every night, every day, etc.).

The treatment information obtainer 114 obtains, as treatment information, the treatment/prescription performed on the patient by the doctor, caregivers and other staff. For example, treatment information is obtained from another server or is obtained by input by a doctor, caregivers or other staff. Examples of treatment information obtained from other servers may include surgical information 332 that can be obtained from the surgical department server 30, medicine information 432 that can be obtained from the medicine department server 40 and medical care information 532 that can be obtained from the first department server 50. Then, the obtained treatment information is stored as treatment information 164.

An example of contents obtained as treatment information will be described. The controller 110 (treatment information obtainer 114) obtains surgical information 332 for a patient as treatment information, thereby acquiring surgical contents of the patient and warning contents after surgery. As a result, the determining processor 116, described later, referring to the obtained treatment information, can determine whether the patient is likely to cause postoperative delirium.

The controller 110 (treatment information obtainer 114) obtains information on medicines for the patient by acquiring the medicine information 432 for the patient. As a result, the determining processor 116, described later, referring to the obtained medicine information, can determine whether the risk the patient would fall down from the bed will increase. If a medicine (e.g., benzodiazepine derivatives) that greatly affects the possibility of falling down from the bed has been administered, the determining processer 116 determines the risk will increase.

The determining processor 116 refers to the patient information 162 and/or the treatment information 164 and determines the risk the patient would fall down from the bed. Then, ways against the risk are determined to reduce the risk and output some ways as proposal information. The processing contents of the second process will be described later. The second process indicates how to determine the proposal information to reduce the risk of falling down from the bed.

The equipment selector 118 selects necessary equipment and devices based on the proposal information output from the determining processor 116. The details of the process (the third process) that the equipment selector 118 selects the equipment currently necessary for the patient in accordance with the content of the proposal information will be described later.

The equipment ordering unit 120 places an order for the equipment selected by the equipment selector 118 on the equipment management server 20. As a result, appropriate equipment for patient's treatment is ordered and provided. Regarding to providing equipment, for example, the equipment ordering unit 120 may make a proposal to the expert such as a doctor, and the equipment may be automatically transported after the controller 110 gets approval from the expert.

The proposing unit 122 makes necessary proposals based on the proposal information output by the determining processor 116. According to the contents of proposal information, the proposing unit 122 displays the proposals necessary for the patient at present on the display unit 150 and displays necessary information to the expert such as a doctor.

For example, the equipment ordered by the equipment ordering unit 120 and the operation terminal allowing the doctor or other experts to operate the recommended equipment may be displayed on the display unit 150. Thus, the expert checks the content and approves the content, then the equipment is ordered.

In addition, the proposing unit 122 may propose use of a room X for the patient together with a wheelchair. In this case, if there is no wheelchair available or when the expert did not approve the use of a wheelchair, the proposing unit 122 may recommend use of another room Y for the patient as an alternative.

The communication unit 130 communicates with external devices and servers. For example, the communication unit can communicate with other servers via a wired LAN such as Ethernet®. Further, the communication unit is a functional unit for communicate with other devices such as the biological information obtainer 12, the patient status detector 14 and terminal devices, and communicates through USB (Universal Serial Bus), Bluetooth® and the like.

The operation unit 140 and the display unit 150 enable the caregivers such as doctors and staff, the medical staff etc., to input instructions and display information. For example, the operation unit 140 and the display unit 150 are integrally configured by a liquid crystal display and a touch panel. When a terminal device 16 can communicate with the management apparatus 10, the terminal device 16 may include these functions of the operation unit 140 and the display unit 150.

The operation unit 140 enables a doctor or expert to input the content of treatment and prescription to the patient, and enables caregivers like a nurse to input the content of treatment. In addition, when, for example, a recommended proposal is displayed on the display unit 150, the professional can check the displayed content, and approve the displayed contents or change or cancel the proposal.

The storage unit 160 stores various programs and various data necessary for the operation of the management apparatus 10. The storage unit 160 is composed of, for example, an SSD (Solid State Drive) that is a semiconductor memory, a HDD (Hard Disk Drive) that is a magnetic disk, and the like.

Further, the above-described patient information 162 and treatment information 164 are stored in the storage unit 160.

### [1.3 Processing Flow]

### [1.3.1 The First Process]

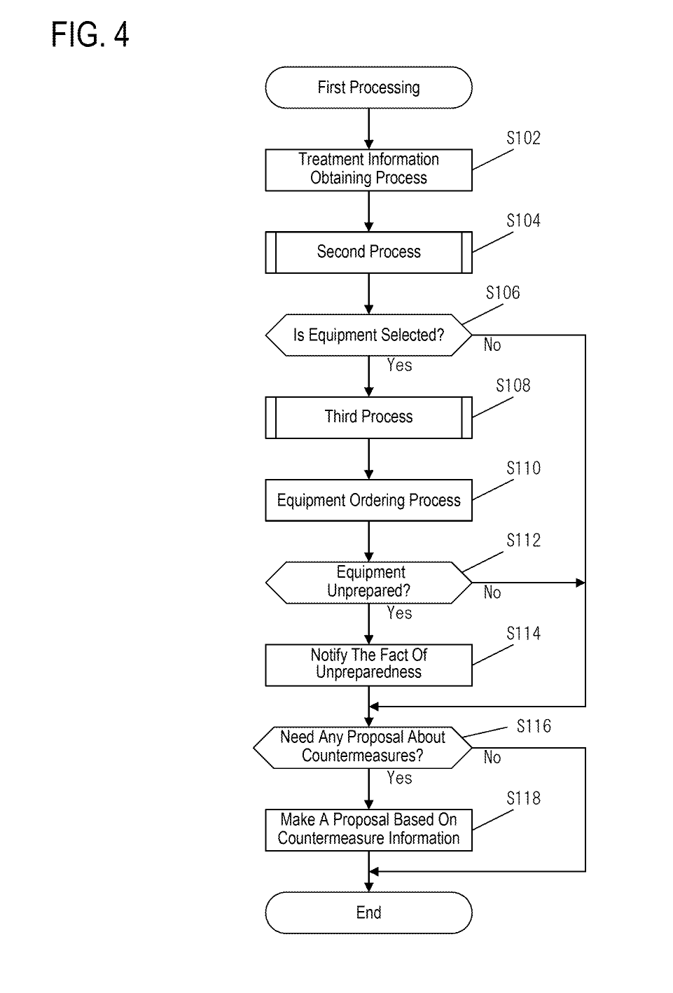

Next, the processing flow of the management apparatus 10 in the present embodiment will be described with reference to FIG. 4. First, the controller 110 (treatment information obtainer 114) executes a treatment information obtaining process (Step S102). Thus, the obtained treatment information is stored as the treatment information 164.

The treatment information obtainer 114 obtains treatment information on the patient from information input by the caregivers or from information stored in other servers. The treatment information obtainer 114 obtains surgical information 332 from the surgical department server 30, obtains medicine information 432 from the medicine department server 40 and obtains medical care information 532 from the first department server 50.

It should be noted that, regarding the treatment information, the treatment information obtainer 114 may obtain necessary treatment information only. That is, if a patient is medicated but has not undergone surgery, the treatment information obtainer 114 obtains the medicine information 432 and the medical care information 532.

Subsequently, the controller 110 (determining processor 116) executes a second process based on the treatment information (Step S104). Here, the detailed operations of the second process effected by the determining processor 116 will be described later. The determining processor 116 executes a second process and outputs proposal information. Then, the controller 110, based on the output proposal information, determines whether at least one of the equipment is needed (Step S106).

Here, when equipment is selected in the proposal information (Step S106, Yes), the controller 110 (the equipment selector 118) executes a third process (Step S108). Based on the proposal information output from the determining processor 116, the equipment selector 118 executes equipment selection to select specific equipment such as sensors, welfare equipment, etc. This third process will be described later.

Information on the selected equipment is output as selected equipment information. For example, as the sensor, detector which evaluates whether the patient is sleeping, detector which can detect whether the patient gets out of the bed, a mat sensor, a string sensor, an infrared sensor, etc. may be selected. In addition, as the welfare equipment, bed whose sections can be relatively low height, electrical bed, assisting devices, handrails which can attach with the bed, cushioning mat which can attach with the bed, can be selected.

Subsequently, the controller 110 (equipment ordering unit 120) executes an equipment ordering process for ordering the selected equipment to the equipment management server 20 (Step S108→Step S110). Here, as the equipment ordering process executed by the equipment ordering unit 120, the following method can be considered.

(1) The equipment ordering unit 120 transmits the selected equipment information to the equipment management server 20. When there is available (suppliable) equipment corresponding to the selected equipment information in the equipment management server 20, the equipment is supplied. At this time, the equipment ordering unit 120 notifies the experts that the equipment has been ordered. When the expert approves it, the equipment is ordered.

(2) When equipment is also managed by servers other than the equipment management server 20 and if there is no available equipment managed by the equipment management server 20, the equipment ordering unit 120 inquires of the servers managing equipment (e.g. the second department server 60) whether or not there is available equipment. When there is available equipment managed by the second department server 60, the equipment ordering unit 120 places an order of the equipment managed by the second department server 60, and then the equipment is supplied. At this time, the equipment ordering unit 120 may supply the selected equipment after coordination with the department that manages the second department server. The coordination of equipment may be done by experts, caregivers and/or equipment management department.

When the equipment can not be prepared (Step S112; Yes), the equipment ordering unit 120 notifies that the equipment can not be prepared (Step S114). The notice may be displayed on the display unit 150 or notified by voice. Further, the equipment ordering unit 120 may transmit the notice to the terminal devices and the like possessed by medical staffs or caregivers. When informing that the equipment can't be prepared, the equipment ordering unit 120 may simply display the fact on the display unit 150 (or the terminal device 16), or may check, once again, whether or not an alternate device is suppliable.

Suppose that, for example, the equipment selected by the equipment selector 118 is an infrared sensor and that the equipment ordering unit 120 can not prepare an infrared sensor, then the equipment ordering unit 120 checks whether or not a string sensor is available as an alternative selected equipment. If the string sensor is available, then the equipment ordering unit 120 displays the situation and places an order for the string sensor.

When it is determined that a proposal should be made as to the proposal information output by the determining processor 116, (Step S116; Yes), the proposing unit 122 makes a proposal based on the contents determined as the proposal (Step S118).

For example, referring to the patient information 162 and the treatment information 164, the proposing unit 122 may determine the location of the hospital room of the patient as the proposal information. Specifically, the proposing unit 122 makes such a proposal so as to select a hospital room close to the nurse station if the patient has just undergone surgery or if the patient is seriously ill. The proposing unit 122 makes proposals by displaying them on the display unit 150 or displaying them on the terminal unit 16.

When rehabilitation programs are provided, the proposing unit 122 may propose a new rehabilitation program.

Further, in the present embodiment, for explanation the first process is performed only once. However, the controller 110 may repeat the first process as necessary. For example, when the proposal is needed, a proposal is made based on proposal information, and then the first process from Step S102 may be repeated. Conversely, when no proposal is needed, the first process from Step S102 may be repeated.

### [1.3.2 The Second Process]

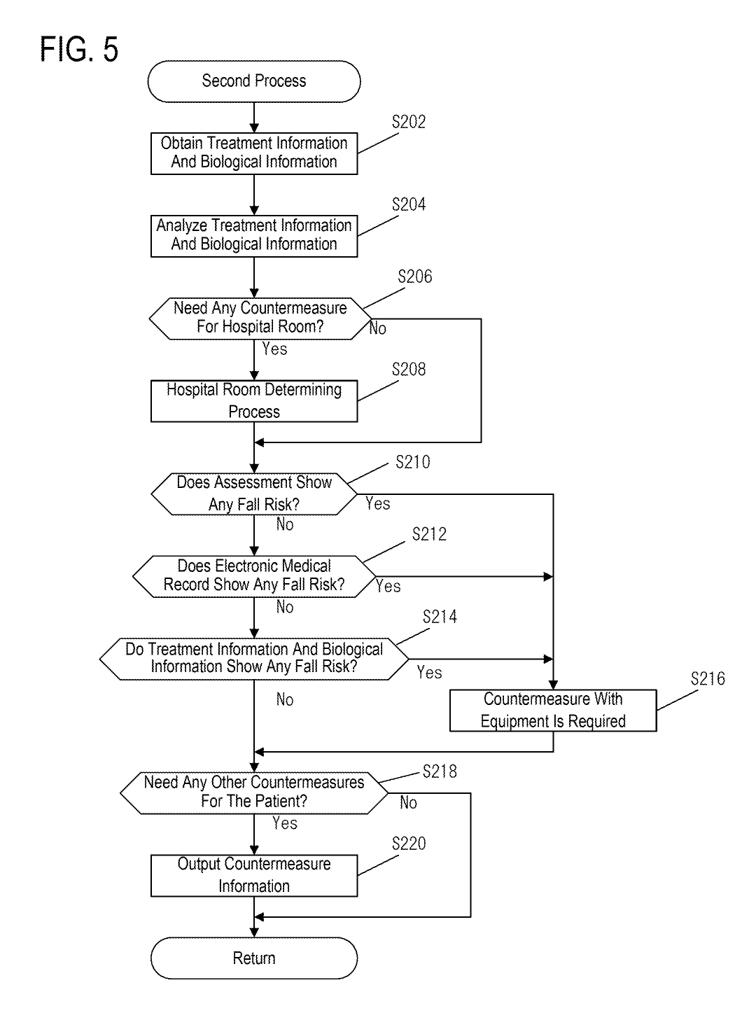

Details of the second process at the above Step S104 in the first process will be described below with reference to FIG. 5.

The determining processor 116 obtains treatment information and biological information (Step S202) and analyzes these information (Step S204). Then, the risk of falling down from the bed of the patient is estimated by analyzing, for example, the obtained surgical information 332, medicine information 432, medical care information 532, and/or biological information such as respiration, heart beat, the status of the patient related to whether the patient is sleeping, depth of the sleeping and the like.

Here, the determining processor 116 determines whether or not a proposal about the room of the patient in the hospital is required (Step S206). Then, if the determining processor 116 determines that proposals about the room of the patient should be needed, the determining processor 116 executes a hospital room determining process (Step S208).

The hospital room determining process determines whether or not there is a risk that the patient will fall down from the bed based on whether the patient is immediately after surgery or based on whether medicine the patient had taken is likely to cause postoperative delirium or the like. It is also judged from the recorded assessment, whether or not the risk or possibility that the patient falls down from the bed is high and whether or not the necessity of care is high to take care of the patient.

When it is determined that the risk of falling down from the bed or the necessity of care is high, the determining processor 116 perform a proposal that the patient should be in or transfer to a room X which is close to the nurse station. Here, if the room X is not vacant, the determining processor 116 performs a proposal that the patient should be in or transfer to a room Y which is the second closest to the nurse station.

Subsequently, when it is determined based on the assessment that there is a risk or relatively high possibility of falling down from the bed (Step S210; Yes), the determining processor 116 determines that at least one of the equipment is needed (Step S216).

When it is determined based on the electronic medical record that there is a risk or relatively high possibility of falling down from the bed (Step S212; Yes), the determining processor 116 determines that at least one of the equipment is needed (Step S216). Here, the proposal is determined based on the electronic medical record, but a proposal may be determined based on an assessment sheet, for example.

Also, when it is determined based on a result of the analysis of the treatment information and/or biological information that there is a risk or relatively high possibility of falling down from the bed (Step S214; Yes), the determining processor 116 determines that at least one of the equipment is needed (Step S216).

Specifically, the determining processor 116 can determine whether or not there is a risk or relatively high possibility of falling down from the bed by checking out the conditions as follows:
(1) Age of the patient: for example, whether or not the patient is 70 years old or older, or whether or not the patient is 9 years old or younger;
(2) history of the patient: whether or not the patient has an experience of falling down from the bed;
(3) Sensory impairments: whether or not the patient has visual impairment and/or hearing impairment;
(4) Dysfunction: whether or not the patient has paralysis, numbness, bone and joint abnormalities etc.;
(5) Activity of the patient: whether or not the patient has muscle weakness of some or part of the patient's body, or whether or not the patient usually uses a wheelchair, walking stick, or walker, or whether or not the patient usually needs assistance if the patient want to move, or whether or not the patient is unsteady on the patients legs (whether the patient can stand by himself or herself), or whether or not the patient is bedridden condition;
(6) Cognitive ability: whether or not the patient has disorientation, clouding of consciousness, confusion, dementia, decline of judgment and understanding, disturbing behaviors, declining memory and re-learning difficulty;
(7) Use of specified medicines: for example, whether or not the patient uses analgesics, narcotics, or sleep stabilizer; and
(8) Excretion: whether or not the patient has urine/fecal incontinence, whether or not the patient is frequent urination, whether or not the patient needs assistance if the patient want to go to the toilet or restroom etc.

When the patient have at least one of troubles and problems related to the above conditions, the patient is treated as the person who has a risk or relatively high possibility of falling down from the bed. Alternatively, the risk or relatively high possibility of falling down from the bed may be evaluated by taking troubles and problems related to a plurality of the above-described conditions into the consideration. For example, the risk or relatively high possibility of falling down from the bed may be determined if there are some troubles and problems in a plurality of conditions or based on the total score by adding up the scores of conditions where the patient has troubles and problems.

Though, in the present embodiment, the specific equipment is selected by the third process to be described later, it may be executed together with the second process.

When other proposals are needed to be taken for the patient, the determining processor 116 outputs proposal information (Step S218; Yes→Step S220).

As an example of other proposals for the patient, the output proposal is to change contexts or schedule of the rehabilitation program if a rehabilitation program is currently scheduled or the patient currently perform the rehabilitation program.

As a method for determining the risk or relatively high possibility of falling down from the bed based on the contents of the electronic medical record described above, the risk may be determined based on the degree of necessity of nursing recorded in the electronic medical record, or may be determined by letting artificial intelligence analyze the contents of the electronic medical record.

If there is no troubles and problems in any conditions, the determining processor 116 may determine that no proposal is required.

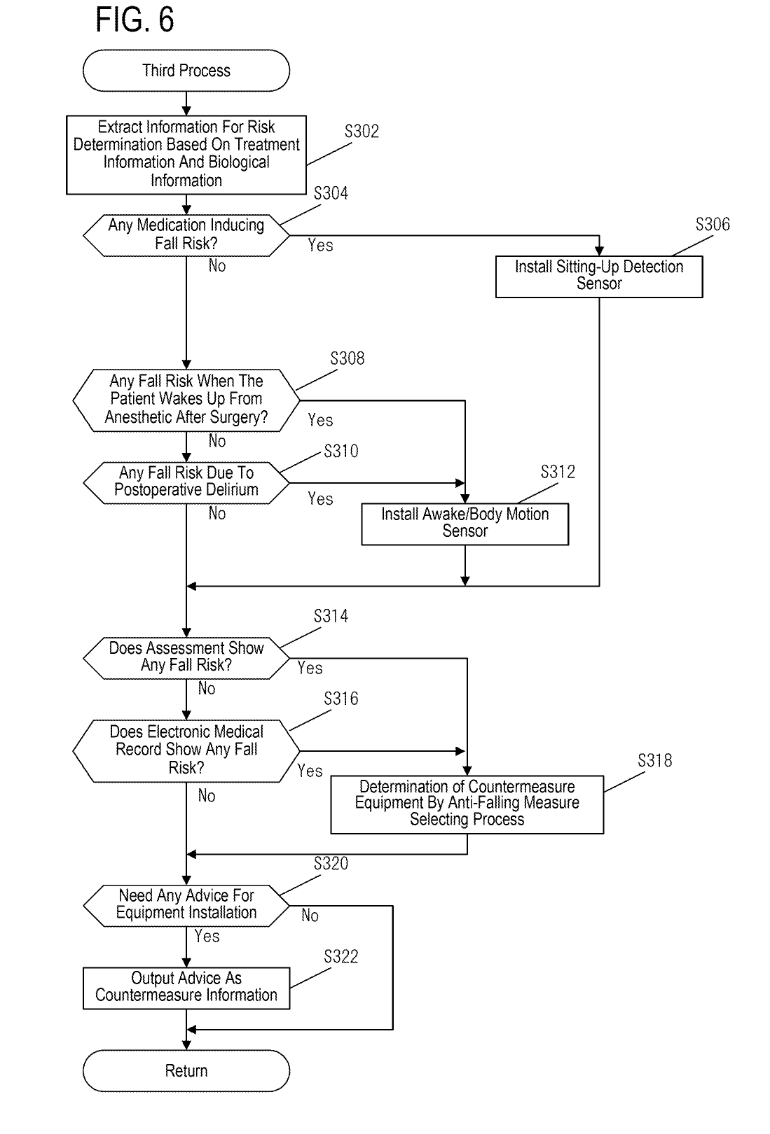

In this embodiment, the hospital room determining process is executed in the second process, but may be executed in the devise selecting process in FIG. 6.

Further, in the present embodiment, for convenience of explanation the process is performed only once. However, the determining processor 116 may repeat the process as necessary. For example, when it is necessary to take proposals, the determining processor 116 may repeat the process from Step S202 after output of the proposal information. Conversely, when no proposal information is output, the process from Step S202 may be repeated.

### [1.3.3 The Third Process]

Next, the third process executed by the equipment selector 118 will be described with reference to FIG. 6. First, the equipment selector 118 extracts information necessary to evaluate the risk or the possibility of falling down from the bed based on the treatment information and/or biological information (Step S302). This step is executed to extract information to be used later by collecting basic information such as the patient's age, medical condition, medicine information and surgical information from the electronic medical record and by analyzing the patient's status from assessment. Further, the patient's heartbeat, respiration etc. are analyzed by acquiring biological information so as to extract information necessary to evaluate the risk or the possibility of falling down from the bed.

The equipment selector 118 determines that whether medicine the patient had taken is likely to cause postoperative delirium based on a result of the analysis of the medicine information 432 (Step S304). If the patient had taken the specified medicine which causes postoperative delirium (Step S304; Yes), the equipment selector 118 decides installation of a sensor for detecting the patient gets up as the proposal information (Step S306). Specifically, the risk or the possibility is decided by matching each type of medicines the patient had taken, with a medicine database or the like, or by judging whether or not medicines causing patient postoperative delirium are included.

In addition, the equipment selector 118 determines from analysis of the surgical information 332 that whether the patient has any risk or relatively high possibility of falling down from the bed. If the equipment selector 118 determines there is the risk of falling down from the bed when the patient wakes up from anesthetic after surgery (Step S308; Yes) or If the equipment selector 118 determines that there is the risk of falling down from the bed due to postoperative delirium (Step S310: Yes), the equipment selector 118 determines installation of a sensor for detecting whether the patient is awake or not by a body motion of the patient as the proposal information (Step S312).

Note that the process in FIG. 6 is merely an example and may be combined as necessary. For example, if the patient had taken the specified medicine which causes postoperative delirium, the equipment selector 118 may install the sensor for detecting whether the patient gets up and also install the sensor for detecting whether the patient is awake or not by a body motion of the patient.

At this time, the equipment selector 118 may change the proposals depending on the medicine which causes postoperative delirium (such as strength of the medicine itself, the likelihood of side effects, combination of prescribed medicines, etc.). Further, the equipment selector 118 may change the proposal according to the patient's attributes (e.g., patient's age, condition and behavior).

The equipment selector 118 may further evaluate the risk or the possibility of falling down from the bed based on the biological information to select equipment. For example, the equipment selector 118, referring to the calculated amount of activity of the patient and the number of times the patient got out of the bed, selects equipment to be installed such as the sensor for detecting whether the patient gets up and/or a sensor for detecting whether the patient gets out of the bed, and changes the thresholds to be notified or alarmed by the selected sensors.

When the equipment selector 118 determines that there is a risk or relatively high possibility of falling down from the bed for the patient based on assessment (e.g., falling and tumbling assessment, medical care environment assessment) (Step S314; Yes), or when the equipment selector 118 determines that there is a risk or relatively high possibility of falling down from the bed for the patient based on the electronic medical record (Step S314; No→Step S316; Yes), the proposed equipment is determined by an anti-falling measure selecting process of the equipment selector 118 (Step S318).

Examples of the methods for selecting countermeasure equipment by the anti-falling measure selecting process of the equipment selector 118 may be listed as follows:

For example, the following criteria are set as conditions.
(1) whether the patient is completely bedridden
(2) whether the patient can keep the posture seating at the edge of the bed for 10 minutes
(3) whether the patient can stand up from a chair or a bed
(4) whether the patient can walk
(5) whether the patient presses the nurse call when the patient gets out of the bed
   The equipment selector 118 selects equipment corresponding to the conditions which the patient has troubles and problems. For example, if, as to the criteria (1), a patient is determined to be completely bedridden, the equipment selector 118 determines that no equipment is selected because there seems to be no risk or possibility of falling down from the bed.

As to the criteria (2), if a patient is unable to keep the posture seating at the edge of the bed for 10 minutes, but always pushes the nurse call when the patient gets out of the bed, equipment such as “electric bed”, two “side-rails”, “sliding sheet”, and “wheelchair” may be selected. Likewise, if a patient is unable to keep the posture seating at the edge of the bed for 10 minutes, and does not push the nurse call when the patient gets out of the bed, the equipment selector 118 determines that equipment such as “sensor for detecting whether the patient gets up”, “bed whose sections can be relatively low height”, “cushioning mat”, three “side-rails”, “sliding seat”, and “wheelchair” may be selected.

On the other hand, if a patient can keep the posture seating at the edge of the bed for 10 minutes and can rise from the chair or the bed, but needs to be watched when the patient is walking, the equipment selector 118 determines that a “sensor for detecting whether the patient gets out of the bed” may be selected. Also if when the patient pushes the nurse call when the patient gets out of the bed, the equipment selector 118 may determine that no “sensor for detecting whether the patient gets out of the bed” is needed.

In this manner, the equipment selector 118 can select necessary equipment according to the state of the patient, the type of medicine and the state of treatment.

Further, the equipment selector 118 outputs necessary sensors or accessories as the proposal information if other sensors or other accessories are required for the patient according to the selected equipment and the state of the patient (Step S320; Yes→Step S322).

For example, by reference to the electronic medical record information, the equipment selector 118 can understand the state of the patient such as which side of the patient is paralyzed. Therefore, if the equipment selector 118 determines that the assisting bar is selected, a location (which side of the bed) the assisting bar should be attached to is output together as the proposal information. Also, when placing a sensor detecting the state of the patient whether the patient is sleeping or is awake, the equipment selector 118 outputs, as the proposal information, the position of the sensor to be attached to the bed.

The proposal information is displayed, for example, on the display unit 150, so that caregivers can confirm the proposal information. Thus, it is possible to install equipment properly and easily.

### [1.3.4 Sequence Diagram]

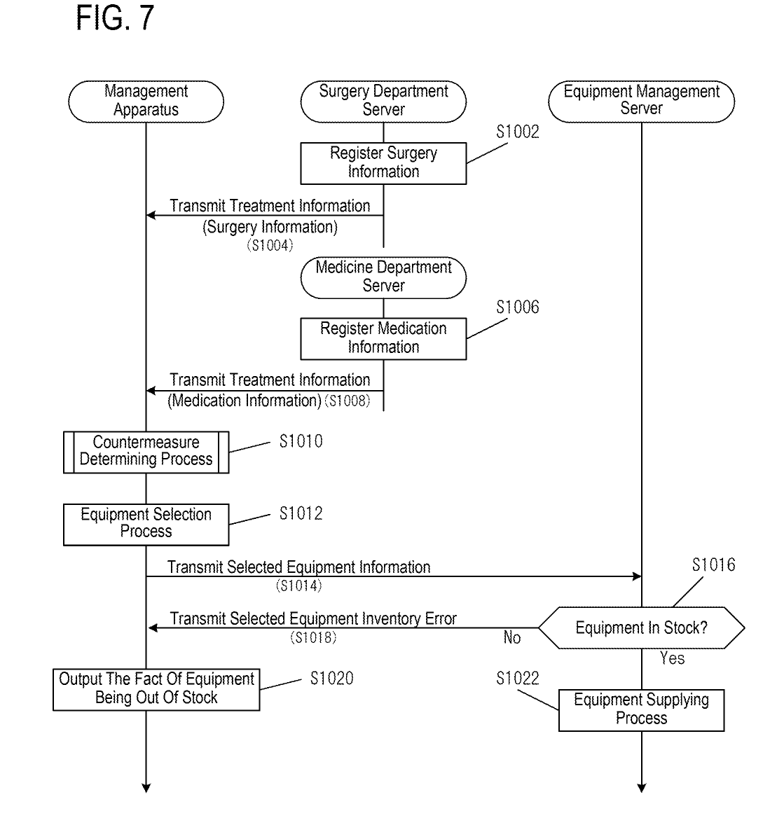

Next, the flow of the entire system will be explained using a sequence diagram in FIG.7. For example, the process of the management apparatus 10 described above may be executed on the management apparatus side to select equipment, or may be triggered by registration of a procedure in another department server.

For example, as shown in FIG. 7, when surgical information is registered in the surgical department server 30 (S1002), the treatment information (surgical information) is transmitted to the management apparatus 10 (S1004). When medicine information is registered in the medicine department server 40 (S1006), the treatment information (medicine information) is transmitted to the management apparatus 10.

In response to this, the management apparatus 10 executes the second process based on the surgical information and the medicine information as the treatment information (S1010) and determines proposal information. Based on the determined proposal information, the management apparatus 10 performs the third process to select necessary equipment (S1012), and transmits the information (selected equipment information) on the selected equipment to the equipment management server 20 (S1014).

Here, when the selected equipment is available (suppliable) in stock (S1016; Yes), the equipment management server 20 executes a distributing process of the selected equipment. Thus, the selected equipment is supplied (S1022).

On the other hand, if there is no equipment in stock (S1016; No), the equipment management server 20 transmits an error that indicates there is no selected equipment in the stock to the management apparatus 10 (S1018). The management apparatus 10 outputs notice that the equipment is not currently in stock (S1020).

As described above, in the department system in the ward, if the patient had taken a medicine which induces components causes the patient postoperative delirium, the order of the medicine is transmitted to the medicine department (medicine department server 40), and the information that the patient had taken the medicine (treatment information) is also transmitted to the management apparatus 10.

The management apparatus 10 selects appropriate equipment based on the proposal information determined based on the treatment information. Accordingly, the proposals such as provision of the sensor for detecting whether the patient gets out of the bed etc. will be ordered together with the order of the medicine.

If a medical treatment the patient is likely to cause postoperative delirium is given or if the patient wakes up from the anesthetic after surgery, the patient has the risk or relatively high possibility of falling down from the bed. Therefore the treatment information is transmitted to the management apparatus 10 when the patient is brought back to the medical ward. Then, the management apparatus 10 determines proposal information based on the treatment information, and selects appropriate equipment. Accordingly, proposal such as provision of the sensor for detecting whether the patient gets out of the bed etc. will be ordered as the patient is brought to the sickroom.

### 2. Second Embodiment

Next, a second embodiment will be described. In the first embodiment, the equipment selector 118 selects equipment based on the proposal information output by the determining processor 116, and the equipment ordering unit 120 orders the selected equipment. In this embodiment, various controls are performed based on proposal information. The system configuration and the functional configuration are the same as those in the first embodiment, so that the differences from the first embodiment will be mainly described.

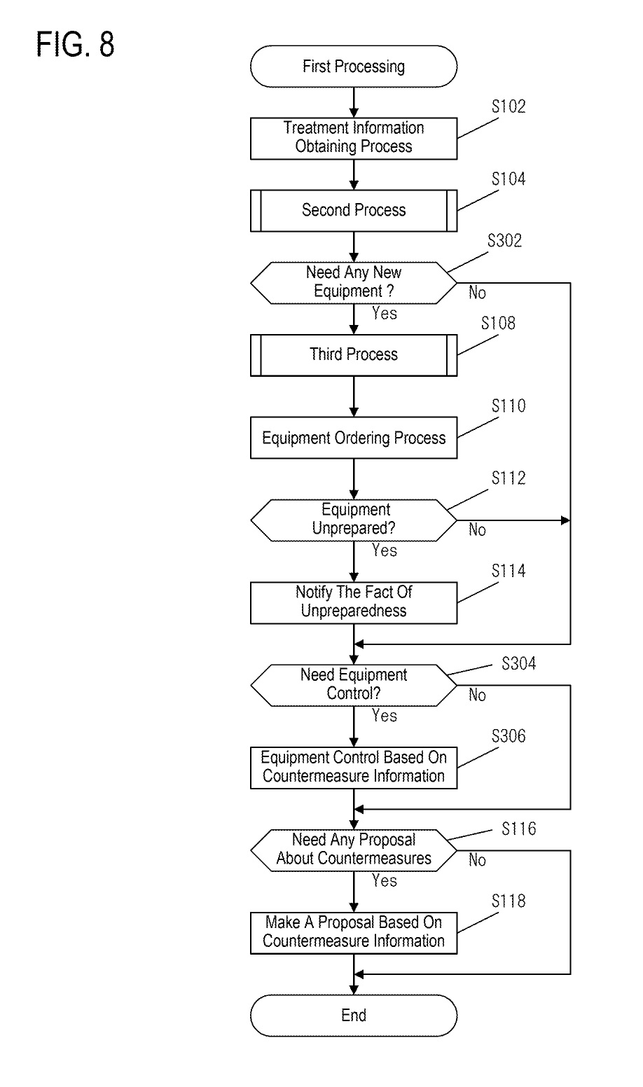

FIG. 8 is a diagram replacing the operation flow of FIG. 4 of the first embodiment. The same procedures are allotted with the same reference numerals.

In the present embodiment, when it is determined that equipment selection is newly necessary at the countermeasure determining process S104, the controller 110 executes an equipment selection process and an equipment ordering process (Step S302; Yes→Step S110).

Here, when the currently installed equipment needs to be controlled based on proposal information, those are controlled based on the proposal information (Step S304; Yes→Step S306).

Specifically, the following control can be considered.
When an electric bed is used, the height of the bed is controlled.
When an air mat is used, the internal pressure of the air mat is controlled.
When the sensor for detecting the patient getting out of the bed is used, control to switch on/off of the sensor for detecting the patient getting out of the bed and control to adjust the sensitivity of the sensor for detecting the patient getting out of the bed are performed.

As described above, according to the present embodiment, it is also possible to control installed equipment based on proposal information.

### 3. Third Embodiment

Next, a third embodiment will be described. In the third embodiment, the determining processor 116 performs a countermeasure determining process, based on not only the treatment information but also other patient information including biological information and the like.

The countermeasure determining process and selection of equipment performed by the determining processor 116 and the equipment selector 118 will be described hereinbelow. This embodiment has the same configurations and features as those in the first embodiment.

### (1) Based on Biological Information

Based on the biological information obtained from the biological information sensor 12, specifically, the real-time conditions such as pulse rate, respiratory rate, body motion, the state of patient (a sleeping state, an awaken state) and the like, the determining processor 116 determines countermeasures. For example, when the patient moves greatly and often turns over in bed when sleeping, the determining processor 116 determines that the risk of falling is high. Then, the equipment selector 118 determines that the sensor for detecting the patient getting out of the bed is necessary and that side fences are necessary in the bed, and selects those as the necessary equipment.

### (2) Based on Patient's Information (Individual Information)

For example, the determining processor 116 performs a countermeasure determining process taking into consideration of the risk of the physical capabilities based on the patient's age and other factors. For example, if the patient is bedridden, no assisting bar is necessary as a countermeasure, so that the determining processor 116 determine that no countermeasure is necessary. On the other hand, when the patient can move, the determining processor 116 determines that countermeasure is necessary, and the equipment selector 118 determines that the handrail is needed.

### (3) Based on Cognitive Risk

For example, the determining processor 116 determines countermeasures in consideration of the number of times the nurse call is pushed by the patient. The determining processor 116 makes different countermeasures for when the patient presses the nurse call frequently and less frequently. Alternatively, the determining processor 116 may change the measures to be made, depending on the duration in which the nurse call is pressed and/or depending on the frequency the nurse call is pressed.

### (4) Based on Action Patterns

For example, taking into consideration whether or not the patient will discharge from the hospital soon, the determining processor 116 changes countermeasures. For example, immediately before discharge, patients often move, which may increase the risk that the patient falls down from the bed. Therefore, the determining processor 116 determines a countermeasure against falls, and the equipment selector 118 determines to install a sensor for detecting a getting-up of the patient.

The contents described in the above embodiments may be used in combination. For example, by combining the treatment information and the patient information (biological information), the determining processor 116 can determine more preferable countermeasures.

### 4. Fourth Embodiment

A fourth embodiment will be described. In the above-described embodiments, the equipment necessary for the patient is selected. The embodiment describes that the equipment once needed can be removed when it is determined based on repeated procedures that the equipment becomes unnecessary.

For example, the process of FIG. 4 is periodically repeated on a patient. For example, the controller 110 grasps the fact that the patient often gets out of the bed and has a great deal of action, in the treatment information obtaining process (Step S102). As a result, the determining processor 116 determines that restraint of the patient is necessary in the countermeasure determining process (Step S104).

In this case, the equipment selector 118 selects a restraint device in the equipment selection process (Step S108). Then, the equipment ordering unit 120 places an order for the equipment in Step S110. Thus, the restraint device is used for the patient.

When the process of FIG. 4 is executed once again, the controller 110 obtains a reduced number of times the patient got out of the bed and a smaller amount of activity at the treatment information obtaining process (Step S102), the determining processor 116 determines that the patient does not need any restraint at the countermeasure determining process (Step S104).

In this case, since no restraint device is necessary in the equipment selection process (Step S108), the equipment selector 118 performs a process for cancelling the current selection of equipment (making the equipment unnecessary). Since no equipment is necessary at Step S110, the equipment ordering unit 120 performs canceling of the installed equipment (for example, removing the equipment, making the staff collect the equipment, and others). Then, the restraint device is removed from the patient, and the equipment management server 20 manages that the restraint is newly available.

As described above, according to the present embodiment, it becomes possible to select necessary and unnecessary equipment for the patient in real time by repeating the determining process a plural number of times.

### 5. Fifth Embodiment

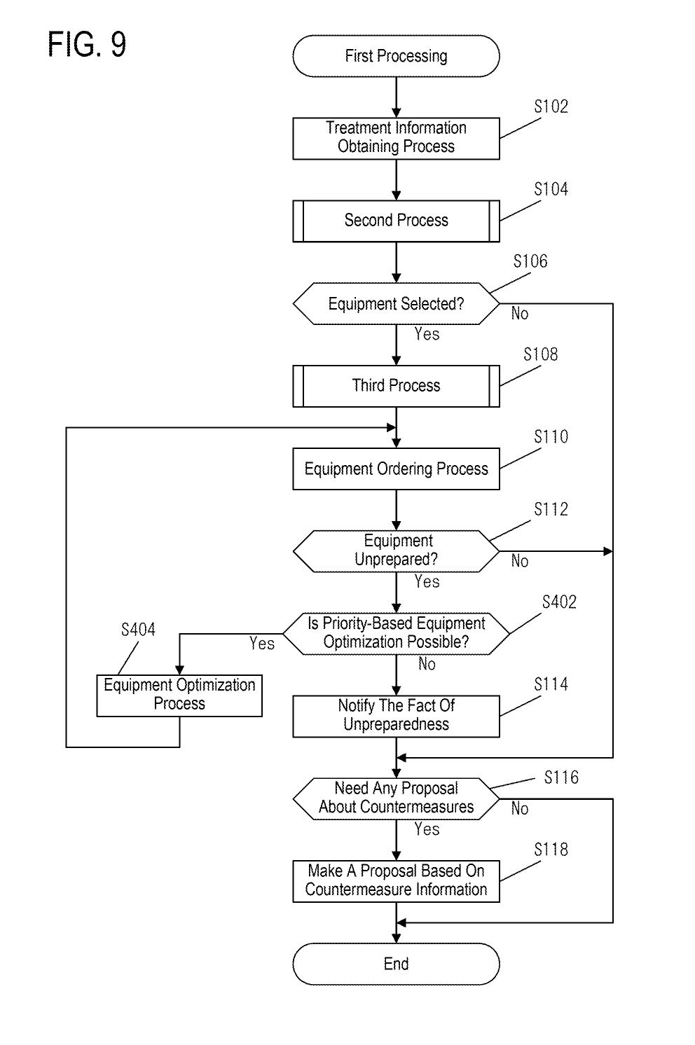

A fifth embodiment will be described. In this embodiment, the processing flow of FIG. 4 of the first embodiment is replaced with that of FIG. 9, and the same procedures are denoted by the same reference numerals with their description omitted.

In the present embodiment, when the equipment ordering unit 120 has ordered equipment (Step S110), but the equipment is not available, the equipment ordering unit 120 determines whether or not equipment optimization according to the priority is possible (Step S402). If use of the equipment can be optimized, the controller 110 (or the equipment management server 20) executes an equipment optimization process. Then, the equipment ordering unit 120 places an order for the equipment once again.

Here, the equipment optimization process means to optimize use of equipment having been used by patients. For example, a group of patients who need a biological information measuring device are prioritized. Specifically, a patient who has to be monitored by awakening detection is given higher priority, whereas a patient who just needs to be monitored by getting-up detection is given lower priority. That is, a biological information measuring device can detect awakening and getting-up of a patient, but it is preferable that the biological information measuring device is used for a patient with higher priority.

When the number of patients requiring the biological information measuring device exceeds the number of the devices, optimization is performed so that patients of higher priority can use the device. For example, suppose that a patient who needs the biological information measuring device with higher priority appears when five patients have currently used the biological information measuring device.

In this case, the controller 110 cancels the current equipment selection of the biological information measurement device for the patient with the lowest priority, and assigns the biological information measurement device to the patient with the highest priority. In addition, a patient whose use of the biological information measuring device has been canceled may be assigned the sensor for detecting the patient getting out of the bed by the controller 110 (equipment selector 118) instead.

As described above, according to the present embodiment, it is possible to appropriately allocate necessary devices according to the priority of patients.

### 6. Sixth Embodiment

A sixth embodiment will be described. The sixth embodiment is an embodiment in which the equipment management server 20 optimizes the total utilization of equipment according to the usage of the equipment in the entire system.

For example, the equipment management information 232 may store information (equipment utilization rate information) such as the used number of each kind of equipment, time and the amount of money. Further, these equipment utilization rate information may be stored for every ward, every floor and every department.

The controller 210 optimizes equipment utilization by referring to the equipment utilization rate information. For example, the controller 210 calculates the average usage number and the maximum usage number of each kind of equipment as the equipment utilization rate information of the equipment for every month.

Based on this, the controller can determine insufficient supply of equipment (wanted equipment) and excessive supply of equipment. Based on this information, it is possible to reallocate the number of equipment to each ward, each floor and each medical department.

For example, suppose that ten sensors for detecting the patient getting out of the bed are allocated to each of the first and second wards and that the first ward uses eight sensors on the average and ten sensors at maximum, whereas the second ward uses two sensors on the average and five sensors at maximum.

In this case, the controller 210 detects that the sensors for detecting the patient getting out of the bed are insufficient in the first ward while an excessive number of sensors are in the second ward, and can transfer four sensors from the second ward to the first ward, whereby it is possible to realize optical distribution of the equipment.

### 7. Variational Example

Although the embodiments have been described above in detail with reference to the drawings, the specific configuration is not limited to the above embodiments, and designs, features and the like without departing from the gist of the present embodiments should be included in the scope of claims.

Further, in the above-described embodiments, processing is executed by the management apparatus 10, but may be realized by installing application software in a terminal device such as a smartphone, tablet and computer. Alternatively, processing may be performed on the server side and the processed result may be returned to the management apparatus.

In addition, in the embodiments, the program operated on each device is a program (a program for causing a computer to function) for controlling the CPU or the like so as to realize the function of the above-described embodiments. Information handled by these devices is temporarily stored in a temporary storage device (for example, RAM) at the time of processing, and thereafter is stored in storage devices such as ROMs, HDDs and SSDs, read out, modified and written in by the CPU as necessary.

To put the product on the market, the program may be stored on a removable storing medium, or may be transferred to a server computer connected to a network such as the Internet or the like. In this case, it goes without saying that the storage device of the server computer is also included in the present invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A management apparatus comprising:
a memory configured to store treatment information on a patient;
an interface unit configured to communicate with an external device; and
a controller configured to obtain the treatment information from the memory, determine a proposal for the patient, based on the treatment information, and order equipment selected based on the proposal to the external device.

2. The management apparatus according to claim 1, wherein the controller is configured to determine the proposal using whether or not there is a risk or relatively high possibility of falling down from the bed.

3. The management apparatus according to claim 1, wherein the external device is a surgical department server, and
the treatment information is information on surgery for the patient.

4. The management apparatus according to claim 1, wherein the external device is a medicine department server; and
the treatment information is information on medicine to the patient.

5. The management apparatus according to claim 3, wherein the external device is a medicine department server; and
the treatment information is information on medicine to the patient.

6. The management apparatus according to claim 3, wherein the information on surgery includes whether the patient is immediately after surgery, and if the controller determines the patient is immediately after surgery, the controller is configured to determine the proposal to use necessary equipment to prevent the patient from falling down from the bed.

7. The management apparatus according to claim 4, wherein if the information on medicine includes a medicine which the patient had taken and causes the patient delirium, the controller is configured to determine the proposal to use necessary equipment to prevent the patient from falling down from the bed.

8. The management apparatus according to claim 5, wherein if the information on medicine includes a medicine which the patient had taken and causes the patient delirium, the controller is configured to determine the proposal to use necessary equipment to prevent the patient from falling down from the bed.

9. The management apparatus according to claim 2, wherein the controller is configured to evaluate the risk using at least one of the following criteria,
(1) Age of the patient,
(2) history of the patient,
(3) Sensory impairments,
(4) Dysfunction,
(5) Activity of the patient,
(6) Cognitive ability,
(7) Use of specified medicines,
(8) Excretion.

10. The management apparatus according to claim 2, wherein the controller is configured to select necessary equipment using at least one of the following criteria,
(1) whether the patient is completely bedridden,
(2) whether the patient can keep the posture seating at the edge of the bed for 10 minutes,
(3) whether the patient can stand up from a chair or a bed,
(4) whether the patient can walk,
(5) whether the patient presses the nurse call when the patient gets out of the bed.
